(19) United States
(12) Patent Application Publication
(10) Pub. No.: US 2016/0305951 A1
(43) Pub. Date: Oct. 20, 2016

(54) BIOMARKER FOR HUMAN EYES AND METHOD THEREOF

(71) Applicant: Po-Kang Lin, Taipei City (TW)

(72) Inventor: Po-Kang Lin, Taipei City (TW)

(21) Appl. No.: 14/845,746

(22) Filed: Sep. 4, 2015

(30) Foreign Application Priority Data
Apr. 16, 2015 (TW) 104112234

(51) Int. Cl. G01N 33/68; C07C 323/58; A61B 3/135; C07K 14/47

(52) U.S. Cl. CPC G01N 33/6815; C07K 14/4737; C07C 323/58; G01N 33/6893; A61B 3/135; G01N 2800/16; G01N 2333/4737

(57) ABSTRACT

The present invention provides a biomarker for detecting and diagnosing polypoidal choroidal vasculopathy (PCV) of human eyes, including levels of hyperhomocysteinemia that are identified, wherein elevated levels of hyperhomocysteinemia are highly associated with PCV of the human eyes. In addition, the present invention further provides a method for detecting and diagnosing PCV of the human eyes.

### CROSS-REFERENCE TO RELATED APPLICATION

This application claims the priority of Taiwan patent application No. 104112234, filed on Apr. 16, 2015, which is incorporated herewith by reference.

### BACKGROUND OF THE INVENTION

1. Field of the Invention

The present invention relates to detection, prediction and diagnosing of polypoidal choroidal vasculopathy (PCV), and more particularly, the present invention relates to PCV of human eyes based on levels of hyperhomocysteinemia to be identified.

2. The Prior Arts

Polypoidal choroidal vasculopathy (PCV) of human eyes is a common disease that results in human blindness, since PCV has newly formed blood vessels which are relatively fragile, can bleed and rupture easily, and may result in malnutrition due to ineffective blood supply. Also, PCV can easily cause atrophy and death of cells.

Currently, the most effective way of diagnosing this disease by the physician is to utilize the Indocyanine Green Angiography (ICGA). The location of PCV is diagnosed through the highly fluorescent region of image, diagnosed according to the other accompanying syndromes, or PCV can be diagnosed at the low fluorescent (light halo) and also diagnosed by drastically varying the brightness from light to dark in the first 5 minutes.

Furthermore, Age-Related Macular Degeneration (AMD) is a common disease that causes elderly blindness in Asia. AMD can be classified as “dry (early) and “wet (late) types. The dry form of AMD has growths such as choroid drusen or geographic atrophy on the Retinal Pigment Epithelium (RPE). These may chronically result in the vision loss. The wet form of AMD causes bleeding and rupturing of blood vessels easily and insufficient nutrition supply due to choroidal neovascularization (CNV), and this may result in further retinal damage. Accordingly, CNV may damage the retinal visual center causing blindness, as well as forming holes in the retina. Therefore, the causes of AMD are generally related to aging, smoking, drinking, diabetes mellitus, hypertension, gene and blue-light exposure.

Homocysteine is a naturally occurring sulfur-containing amino acid that is produced during the metabolism of methionine. High plasma levels of homocysteine have been identified as an independent risk factor of vascular diseases, such as cardiovascular disease as well as stroke, dementia and Alzheimer's disease. Elevated plasma homocysteine levels are also observed in patients with retinal vascular occlusive diseases, pseudoexfoliative glaucoma and diabetic retinopathy. Similarly, patients with AMD may also present with elevated plasma homocysteine levels.

C-Reactive Protein (CRP) is a systemic inflammatory biomarker and a risk factor of cardiovascular disease. Japanese researcher found a significant association between elevated high sensitivity C-Reaction Protein (hsCRP) and PCV, and concluded that the inflammatory processes were involved in the pathogenesis of PCV.

Therefore, the real solution for the currently existing problems resides in how to identify that hyperhomocysteinemia is associated with PCV and might play a role in the pathogenesis of PCV.

### SUMMARY OF THE INVENTION

In view of the foregoing drawbacks of the prior art, the present invention provides a biomarker for detecting or diagnosing polypoidal choroidal vasculopathy (PCV) of human eyes, including levels of hyperhomocysteinemia that are identified, wherein elevated levels of hyperhomocysteinemia are highly associated with PCV of the human eyes.

In addition, the present invention further provides a method for detecting or diagnosing PCV of human eyes, including the steps of: identifying levels of hyperhomocysteinemia of a plurality of biological samples of the human eyes, and comparing the levels of hyperhomocysteinemia with controls of hyperhomocysteinemia, wherein, elevated levels of hyperhomocysteinemia are highly associated with PCV of the human eyes.

Preferably, data of the plurality of biological samples may include age, gender, lifestyle determinants, smoking, alcohol consumption, medical history, hypertension history, diabetes mellitus history, coronary artery disease history and cerebrovascular event. However, the present invention is not limited hereto.

Preferably, the levels of hyperhomocysteinemia are identified by the following analytical methods: a method of total plasma Homocysteine analysis, a method of high sensitivity C-Reactive Protein (hsCRP) analysis and a method of statistical analysis. However, the present invention is not limited hereto.

Preferably, the method of statistical analysis may use a multivariable logistic regression model to determine relevance between levels of plasma homocysteine and hsCRP and PCV.

Preferably, the method for detecting and diagnosing PCV of the human eyes may further includes a best-corrected visual acuity examination, a slit lamp bio-microscopy examination and a fundoscopy examination of the human eyes.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) depict only the preferred embodiments of the invention and shall not be considered as limitations to the scope of the present invention. Modifications of the shape of the present invention shall be considered within the spirit of the present invention.

The following preferred embodiments of the present invention describe a biomarker for detecting and diagnosing polypoidal choroidal vasculopathy (PCV) of human eyes.

### Study Subjects

According to the embodiments of the present invention, patients diagnosed with PCV were recruited. The control group included subjects who were undergoing routine physical examinations or follow-up for diseases other than age-related macular degeneration (AMD), retinal vascular disease, diabetic retinopathy or glaucoma. Each case was then matched with one control subject on age and gender. All study subjects were of Chinese ethnicity.

According to the embodiments of this invention, all participants received the a complete ophthalmic examination consisting of best-corrected visual acuity examination, slit lamp bio-microscopy examination and fundoscopy examination. In addition, according to the embodiments of the present invention, the fundus photography with fluorescein angiography (FA) taken with a fundus camera (CF-60 UD, Cannon Inc., Tokyo, Japan) and ICGA (Heidelberg Retinal Angiography II, Heidelberg Engineering, Heidelberg, Germany) were performed through dilated pupils in all case subjects. The retinal images were all reviewed by tow retinal specialist (P.K.L. and J.H.L.) in a masked fashion. Only subjects with characteristic poly-like choroidal vessel dilatation (polypoidal lesions or polyps) on ICGA were diagnosed to have PCV. Locations of PCV was divided into extrafoveal, juxtafoveal (within 200 μm of the fovea), subfoveal and peripapillary (within one disc diameter of the optic disc) areas.

In the embodiments of the present invention, data all the participants include age, gender, smoking, alcohol consumption, lifestyle determinants, medical history, hypertension history, diabetes mellitus (DM) disease, coronary artery disease, cerebrovascular event, renal dysfunctions, etc. However, they were not limited with this invention.

### Total Plasma Homocysteine Analysis

In the embodiments of the present invention, fasting venous blood samples were obtained in the sitting position and collected in test tubes containing heparin. Subsequently, the plasma homocysteine levels were measured by automated chemiluminescent (ADVIA Centaur system, Siemens, East Walpole, Mass., USA) with sensitivity of 0.5 μmol/L and a total coefficient of variation (CV) of 6.8% at 4.9 μmol/L, 3.9% in 61.6 μmol/L. In addition, the above-mentioned assay correlates well with other assays including fluorescent polarization immunoassay and high-performance liquid chromatography (HPLC).

High Sensitivity C-Reactive Protein (hsCRP) Analysis

In the embodiments of the present invention, venous blood samples were obtained and collected in the serum separation tubes. Serum hsCRP was measured by rate nephelometry on an automated nephelometer (Immage 800, Beckman Coulter, Fullerton, Calif., USA). This hsCRP assay, with analytical sensitivity of 0.2 mg/L and a total coefficient of variation (CV) of 5.17% in 0.79 mg/L and 3.8% in 13.4 mgl/L has been shown to correlate well with other commonly used assays.

In the embodiments of the present invention, hyperhomocysteinemia and elevated hsCRP were defined as levels above the 95^{th} percentile of the control group.

### Statistical Analysis

In the method of statistical analysis of the present invention, continuous and categorical variables in demographic and medical characteristics were compared between PCV cases and control subjects using Student's t-test and Pearson's chi-square test, respectively. Plasma homocysteine and serum hsCRP levels were presented as median (interquartile range) and compared to controls using Mann-Whitney U test because homocysteine and hsCRP levels were not normally distributed.

In the method of statistical analysis of the present invention, multivariable logistic regression models were used to evaluate whether the PCV was associated with plasma homocysteine or serum hsCRP. All odds ratios (ORs) were adjusted for age, gender, lifestyle factors (smoking and alcohol consumption) and medical histories (hypertension, DM, coronary artery disease and cerebrovascular event).

Furthermore, in the method of statistical analysis of the present invention, SPSS for Windows version 18 (SPSS Inc., Chicago, Ill., USA) was used for these calculations. All reported P-values were based on two-tailed test, and a P-value of less than 0.05 was considered to be statistically significant.

### Results

One hundred and twenty-four case subjects were enrolled initially. Three patients were excluded due to renal dysfunction and two were excluded due to revised diagnosis of CNV with AMD. Therefore, finally, a total of 190 patients with PCV matched controls were enrolled.

Table 1 shows characteristics of patients with polypoidal choroidal vasculopathy, control groups and P values.

As shown in Table 1, the mean age was 72.1±13.0 years in the PCV group and 69.3±10.9 years in the control group, with a male preponderance (74.8%) in both groups. There were no statistically significant differences in age, gender, hypertension, DM, coronary artery disease, cerebrovascular event and lifestyle factors (including smoking and alcohol consumption between case subjects and controls).

TABLE 1
Characteristics of patients with polypoidal choroidal vasculopathy, control
groups and P values

| | PCV (n = 119)) | | Control (n = 119) | | P Value |
|---|---|---|---|---|---|
| Age | 72.1 ± 13.0 | (36.0-93.0) | 69.3 ± 10.9 | (36.0-94.0) | 0.07 |
| Gender (% male) | 89 | (74.8%) | 89 | (74/8%) | 1.00 |

Medical history

| | | | | | |
|---|---|---|---|---|---|
| Hypertension | 56 | (47.1%) | 54 | (45.4%) | 0.80 |
| Diabetes mellitus | 26 | (21.8%) | 28 | (23.5%) | 0.76 |
| Coronary artery disease | 21 | (17.6%) | 19 | (16.0%) | 0.73 |
| Cerebrovascular event | 5 | (4.2%) | 3 | (2.5%) | 0.47 |

Lifestyle determinants

| | | | | | |
|---|---|---|---|---|---|
| Smoking (current/former) | 8 | (6.7%) | 4 | (3.4%) | 0.24 |
| Alcohol consumption | 2 | (1.7%) | 0 | (0%) | 0.16 |

Table 2 shows plasma homocysteine and serum C-reactive protein levels in overall and different genders of patients with polypoidal choroidal vasculopathy and control groups.

As shown in Table 2, of the median plasma homocysteine level was significantly higher in the PCV group (median: 12.20 μmol/L; interquartile range: 9.67-16.66 μmol/L) than in the control group (median: 9.80 μmol/L; interquartile range: 8.13-11.26 μmol/L; p<0.001). The evaluation of homocysteine can be further categorized into the subgroup with DM and without DM. The plasma homocysteine level of PCV patients with DM was significantly higher than the control groups with DM (p=0.001). In addition, the plasma homocysteine level of PCV patients without DM was significantly higher than the control groups without DM (p<0.001). of the median serum hsCRP level was slightly higher in the PCV group (median: 0.16 mg/dl; interquartile range: 0.06-0.30 mg/dl) than in the control group (median: 0.11 mg/dl; interquartile range: 0.06-0.25 mg/dl; p=0.07).

Additionally, the 95^{th} percentile of the homocysteine level in the control group was 13.26 μmol/L. Of the 119 PCV patients, 47 (39.5%) patients exceeded the cutoff value, compared with 5 of 119 (4.2%) patients in the control group (p<0.001). Furthermore, the 95^{th} percentile of hsCRP in the control group was 0.70 mg/dl. In the PCV group, 13 (11.1%) of 118 patients exceeded the cutoff value, as compared with 6 (5.4%) of the 113 patients in the control group (p=0.12).

TABLE 2
Plasma homocysteine and serum C-reactive protein levels in overall and
different genders of patients with polypoidal choroidal vasculopathy
and control groups
Overall

| | PCV (n = 119) | Control (n = 119) | P Value |
|---|---|---|---|
| Homocysteine | 12.20 (9.67-16.66) | 9.80 (8.13-11.26) | <0.001 |
| (μmol/L) | | | |
| hsCRP | 0.16 (0.06-0.30) | 0.11 (0.06-0.25) | 0.07 |

Males

| | PCV (n = 89) | Control (n = 119) | P Value |
|---|---|---|---|
| Homocysteine | 13.91 (11.43-17.65) | 10.48 (8.86-11.91) | <0.001 |
| (μmol/L) | | | |
| hsCRP | 0.17 (0.06-0.30) | 0.11 (0.06-0.24) | 0.14 |

Females

| | PCV (n = 30) | Control (n = 30) | P Value |
|---|---|---|---|
| Homocysteine | 9.29 (7.70-11.11) | 8.18 (7.31-9.00) | 0.02 |
| (μmol/L) | | | |
| hsCRP | 0.15 (0.06-0.42) | 0.09 (0.06-0.26) | 0.22 |

Table 3 shows the distribution of polypoidal choroidal vasculopathy cases, control groups and P-values with tertiles of homocysteine and hsCRP levels.

As shown in Table 3, after stratified by genders, both males and females of the PCV group had the significantly higher plasma homocysteine levels (p<0.001 in male; and p=0.02 in female) and portions of hyperhomocysteinemia (p<0.001 in male; and p=0.02 in female), compared to those of the controls. The results of hsCRP levels were generally insignificant except for the higher proportions of elevated hsCRP in female (26.7% vs. 3.4% in the controls, p=0.01) (as shown in Table 2). If stratified the homocysteine levels into tertile, significantly higher proportion of PCV patients had homocysteine levels in the higher tertile, compared with the controls (67.2% vs. 32.8% in controls, p<0.01).

TABLE 3
Distribution of polypoidal choroidal vasculopathy cases, control groups and
P-values with tertiles of homocysteine and hsCRP levels.

| | | | |
|---|---|---|---|
| | | Multivariable-adjusted OR | |
| PCV (n = 119) | Control (n = 119) | (95% CI) | P Value |

Tertile of homocysteine (μmol/L):

| | | | | |
|---|---|---|---|---|
| 1 (≦8.62) | 16 (13.5%) | 40 (33.6%) | 1.00 (reference) | |
| 2 (8.62-10.81) | 23 (19.3%) | 40 (33.6%) | 1.99 (0.83-4.77) | 0.12 |
| 3 (>10.81) | 80 (67.2%) | 39 (32.8%) | 8.84 (3.68-21.21) | <0.001) |

Tertile of hsCRP (mg/dl)

| | | | | |
|---|---|---|---|---|
| 1 (<0.07) | 34 (29.1%) | 38 (33.9%) | 1.00 (reference) | |
| 2 (0.07-0.17) | 26 (22.2%) | 36 (32.2%) | 0.77 (0.38-1.55) | 0.46 |
| 3 (>0.17) | 57 (48.7%) | 38 (33.9%) | 1.71 (0.91-3.24) | 0.10 |

Regarding the aspect of the multivariable logistic regression analysis, after adjusting for age, gender, hypertension, DM, coronary artery disease, smoking and alcohol consumption, it was observed that elevated plasma homocysteine levels were significantly associated with an increasing risk of PCV (OR, 1.54; 95% confidence interval (CI), 1.33-1.79; p<0.001). Specifically, in the regression model as shown in the Table 3, patients in the highest tertile of homocysteine had an approximately 9-fold increased risk of PCV (OR, 8.84; 95% CI, 3.68-21.21; P<0.001).

Therefore, according to the embodiments of the present invention, the biomarker for detecting and diagnosing PCV of human eyes may include levels of hyperhomocysteinemia that are identified, wherein, elevated levels of hyperhomocysteinemia are highly associated with PCV of the human eyes.

In addition, the present invention further provides a method for detecting and diagnosing PCV of human eyes. The method for detecting and diagnosing PCV of human eyes of the present invention may include the following steps: identifying levels of hyperhomocysteinemia of biological samples of the human eyes; and comparing the levels of hyperhomocysteinemia with controls of hyperhomocysteinemia, wherein elevated levels of hyperhomocysteinemia are highly associated with PCV of the human eyes.

According to the embodiments of the present invention, data of the plurality of biological samples may include age, gender, lifestyle determinants, smoking, alcohol consumption, medical history, hypertension history, diabetes mellitus history, coronary artery disease history and cerebrovascular event. However, the present invention is not limited hereto.

According to the embodiments of the present invention, the levels of hyperhomocysteinemia may be identified by the following analytical methods: a method of total plasma homocysteine analysis, a method of high sensitivity C-reactive protein (hsCRP) analysis and a method of statistical analysis. However, the present invention is not limited hereto.

According to the embodiments of the present invention, the method of statistical analysis may utilize a multivariable logistic regression model to determine relevance between levels of plasma homocysteine and hsCRP and PCV.

According to the embodiments of the present invention, the method for detecting and diagnosing PCV of human eyes of the present invention may further includes: a best-corrected visual acuity examination, a slit lamp bio-microscopy examination and a fundoscopy examination of the human eyes.

Therefore, according to the results of the present invention, the present invention shows that the elevated levels of plasma homocysteine and serum hsCRP are highly associated with PCV of the human eyes.

The above preferred embodiments describe the principle and effect of the present invention, but are not limited to the present invention. It will be apparent to a person ordinarily skilled in the art that various modifications and variations can be made to the disclosed embodiments. It is intended that the specification and examples be considered as exemplary embodiments only, with a true scope of the disclosure being indicated by the following claims and their equivalents.

What is claimed is:

1. A biomarker for detecting and diagnosing polypoidal choroidal vasculopathy (PCV) of human eyes, comprising:
levels of hyperhomocysteinemia that are identified, wherein elevated levels of hyperhomocysteinemia are highly associated with PCV of the human eyes.

2. The bio-marker for detecting and diagnosing PCV of human eyes according to claim 1, wherein the levels of hyperhomocysteinemia are identified by the following analytical methods: a method of total plasma homocysteine analysis, a method of high sensitivity C-reactive protein (hsCRP) analysis and a method of statistical analysis.

3. The bio-marker for detecting and diagnosing PCV of human eyes according to claim 2, wherein the method of statistical analysis uses a multivariable logistic regression model to determine relevance between levels of plasma homocysteine and hsCRP and PCV.

4. The bio-marker for detecting and diagnosing PCV of human eyes according to claim 1, further comprising: a best-corrected visual acuity examination, a slit lamp biomicroscopy examination and a fundoscopy examination of the human eyes.

5. A method for detecting and diagnosing polypoidal choroidal vasculopathy (PCV) of human eyes, comprising the steps of:
identifying levels of hyperhomocysteinemia of a plurality of biological samples of the human eyes; and
comparing the levels of hyperhomocysteinemia with controls of hyperhomocysteinemia, wherein elevated levels of hyperhomocysteinemia are highly associated with PCV of the human eyes.

6. The method for detecting and diagnosing PCV of human eyes according to claim 5, wherein data of the plurality of biological samples include age, gender, lifestyle determinants, smoking, alcohol consumption, medical history, hypertension history, diabetes mellitus history, coronary artery disease history and cerebrovascular event.

7. The method for detecting and diagnosing PCV of human eyes according to claim 5, wherein the levels of hyperhomocysteinemia are identified by the following analytical methods: a method of total plasma homocysteine analysis, a method of high sensitivity C-reactive protein (hsCRP) analysis and a method of statistical analysis.

8. The method for detecting and diagnosing PCV of human eyes according to claim 7, wherein the method of statistical analysis uses a multivariable logistic regression model to determine relevance between levels of plasma homocysteine and hsCRP and PCV.

9. The method for detecting and diagnosing PCV of human eyes according to claim 5, further comprising: a best-corrected visual acuity examination, a slit lamp biomicroscopy examination and a fundoscopy examination of the human eyes.
